SEQUENCE LISTING (10 sequences)

SEQ ID NO: 1 - protein, 598 aa, MUS musculus
MPCVQAQYGSSPQGASPASQSYSYHSSGEYSSDFLTPEFVKFSMDLTNTEITATTSLPSFSTFMDNYSTGYDVKPPCLYQMPLSGQQSSIKVEDIQMHNYQQHSHLPPQSEEMMPHSGSVYYKPSSPPTPSTPSFQVQHSPMWDDPGSLHNFHQNYVATTHMIEQRKTPVSRLSLFSFKQSPPGTPVSSCQMRFDGPLHVPMNPEPAGSHHVVDGQTFAVPNPIRKPASMGFPGLQIGHASQLLDTQVPSPPSRGSPSNEGLCAVCGDNAACQHYGVRTCEGCKGFFKRTVQKNAKYVCLANKNCPVDKRRRNRCQYCRFQKCLAVGMVKEVVRTDSLKGRRGRLPSKPKSPQDPSPPSPPVSLISALVRAHVDSNPAMTSLDYSRFQANPDYQMSGDDTQHIQQFYDLLTGSMEIIRGWAEKIPGFADLPKADQDLLFESAFLELFVLRLAYRSNPVEGKLIFCNGVVLHRLQCVRGFGEWIDSIVEFSSNLQNMNIDISAFSCIAALAMVTERHGLKEPKRVEELQNKIVNCLKDHVTFNNGGLNRPNYLSKLLGKLPELRTLCTQGLQRIFYLKLEDLVPPPAIIDKLFLDTLPF

SEQ ID NO: 2 - DNA, 19 bp, Artificial sequence
  Oligonucleotide probe
tttaccctcgaagccgaag

SEQ ID NO: 3 - DNA, 19 bp, Artificial sequence
  Oligonucleotide probe
tgtatgctaagcgcagaac

SEQ ID NO: 4 - DNA, 20 bp, Artificial sequence
  Oligonucleotide probe
cggtggaactttgacttcgt

SEQ ID NO: 5 - DNA, 20 bp, Artificial sequence
  Oligonucleotide probe
gagtgcaagacagcgacaag

SEQ ID NO: 6 - DNA, 20 bp, Artificial sequence
  Oligonucleotide probe
ccgaggaggaagatgtcaaa

SEQ ID NO: 7 - DNA, 20 bp, Artificial sequence
  Oligonucleotide probe
aaattccacttgcgctgact

SEQ ID NO: 8 - DNA, 20 bp, Artificial sequence
  Oligonucleotide probe
gagagaacttgctgggcatc

SEQ ID NO: 9 - DNA, 20 bp, Artificial sequence
  Oligonucleotide probe
gctttacaccttgggaccag

SEQ ID NO: 10 - DNA, 31 bp, Artificial sequence
  Oligonucleotide probe
agcttgagttttaaaaggtcatgctcaattt

(19) United States
(12) Patent Application Publication
(10) Pub. No.: US 2004/0235096 A1
(43) Pub. Date: Nov. 25, 2004

(54) Method for regulating dopamine producing cells

(76) Inventors: Thomas Perlmann, Stockholm (SE); Joseph Bertrand, Stockholm (SE)

Correspondence Address: FULBRIGHT & JAWORSKI, LLP, 666 FIFTH AVE, NEW YORK, NY, 10103-3198, US

(21) Appl. No.: 10/487,176

(22) PCT Filed: Aug. 26, 2003
(86) PCT No.: PCT/US03/26687

Related U.S. Application Data
(60) Provisional application No. 60/408,132, filed on Aug. 26, 2002.

(51) Int. Cl. C07K 14/705; C07H 21/04

(52) U.S. Cl. 435/69.1; 435/320.1; 435/325; 530/350; 536/23.5

(57) ABSTRACT

Nurr1 derivatives are disclosed. Also disclosed is a method for inducing differentiation of dopamine producing neurons and a method for regulating expression of p57 ^{kip2} via the derivatives

### RELATED APPLICATIONS

This application is a continuation in part of application Ser. No. 60/408,132, filed Aug. 26, 2002, incorporated by reference in its entirety.

### FIELD OF THE INVENTION

This invention relates to the interaction of Nurr1 and derivatives thereof with other molecules, and uses thereof.

### BACKGROUND AND PRIOR ART

Cellular diversification in the central nervous system (“CNS” hereafter), depends upon a strictly regulated interlocking set of signaling events which results, ultimately, in the coordinated expression of region and cell type specific transcription factors. For review articles, see Jessell, et at., Curr. Opin Neurobiol. 10: 599-611. (2000), Briscoe, et al, Curr. Opin. Neurobiol. 11:43-49 (2001). An understanding of how, e.g., transcription factors generate cell diversity in the development of the CNS is of interest for, e.g., treatment of CNS related disorders.

A population of neurons that are located in the ventral midbrain synthesize and release the catecholamine neurotransmitter “dopamine,” which will be abbreviated as “DA” hereafter. Midbrain DA neurons, along with their rostral innervation targets, constitute the major dopaminergic pathways, and are involved in the regulation and control of, e.g., motor integration, cognition, award mechanisms, and memory processing. See Perrone-Capano, et al., Int. J. Dev. Biol. 44: 679-687 (2000). An additional reason why DA cells are important, clinically, is that these cells degenerate in patients with CNS disorders like Parkinson's Disease, and influence processes that are implicated in schizophrenia and other disorders. See, e.g., Dunnett, et al., Nature 399: A32-39 (1999); Bassett, et al., Can. J. Psychiatry 46: 131-137 (2001).

DA cells are generated in the ventral floor of the embryonic midbrain. See Hynes, et al., Curr. Op. Neurobiol. 9: 26-36 (1999). Early signaling by the factors known as “Sonic hedgehog” and “fibroblast grow factor 8,” contribute to patterning events, as well as the establishment of a proliferating, dopaminergic progenitor cell population which expresses retinaldehyde dehydrogenase I (“Raldh/AHD2”). See Hynes, et al., Neuron 15:33-44 (1995); Ye, et al., Cell 93: 755-766 (1998), Wallén, et al., Exp. Cell Res. 253: 737-746 (1999). As the cells stop proliferating, they begin to express the molecule known as Nurr1, or “NR4A2.” “Nurr1” as it will be referred to hereafter is a member of the nuclear receptor family. See Law, et al., Mol. Endocrinol. 6(12): 2129-35 (1992) and Law, et al., NCBI Accession No. A46225, both incorporated by reference.

The murine Nurr1 sequence disclosed by Law, et al. is presented herein as SEQ ID NO: 1. It should be noted that derivatives of Nurr1 discussed herein are intended to also include the human Nurr1 sequence, which differs from the murine Nurr1 sequence by only three residues at positions 131, 134 and 354. See Strausberg, et al., NCBI Accession No. AAH09288, also incorporated by reference. Positions 131, 134 and 354 are t, g and e in the human Nurr1 sequence, while positions 131, 134 and 354 are s, s and d in the murine Nurr1 sequence. Nurr1 as used herein refers to all forms of the molecule, regardless of species (e.g., mammalian, human, murine, primate and other animal species), as well as all isoforms such as those disclosed in public databases, e.g. GenBank.

Nurr1 has been shown to be essential for midbrain DA neuron development. To elaborate, Nurr1 knockout animals lack tyrosine hydroxylase (TH), as well as other dopaminergic characteristics. See Zetterstrom, et al., Science 276: 248-250 (1997); Castillo, et al., Mol. Cell Neurosci. 11: 36-46 (1998); Saucedo-Cardenas, et al., Proc. Natl. Acad. Sci. USA 95: 4013-4018 (1998). Further, Nurr1 is required for sustained expression of DA cell specific genes, normal cell migration, target area innervation, and cell survival. See, e.g., Saucedo-Cardenas, et al., supra; Wallén, et al., Exp. Cell Res. 253: 737-746 (1999). Wallén, et al., Mol. Cell Neurosci 18: 649-663 (2001).

Other transcription factors are involved in DA cell development including the homeodomain containing transcription factors Engrailed 1 and 2. (“En1” and “En2”), and Lmx1b). In contrast to Nurr1, however, these proteins appear to influence more global patterning events in the developing midbrain. See Joyner, et al., Science 251: 1239-1243 (1991); Wurst, et al., Development 120: 857-887 (1994); Smidt, et al., Nat. Neurosci. 3: 337-341 (2000); Simon, et al., J. Neurosci. 21: 3126-3134 (2001) What is common to all of these factors, however, is that the genes they regulate which contribute to DA cell development remain unidentified.

Cellular differentiation, and withdrawal from the cell cycle, are tightly coordinated processes, especially in developing embryos. Several regulatory mechanisms that are essential for the regulation of the cell cycle have been shown to influence cellular differentiation. See Chellappan, et al., Curr. Top. Microbiol. Immunol. 227: 57-103 (1998).

One important mechanism for cell cycle control involves the inhibition of cyclin dependent Kinases, or “CDKs” by CDK inhibitors, or “Ckis.” See, e.g., Vidal, et al., Gene 247: 1-15 (2000). The Cip/Kip family of Clds consists of p21 ^{Cip1}, p27 ^{Kip1}, and p57 ^{Kip2}. They are involved in cell cycle exit and differentiation of various tissues in vivo. See, e.g., Chellapan, et al., supra. Of these, only p57 ^{Kip2} has been shown to play an essential role during embryogenesis that cannot be compensated for by other Ckis. For example, p57 ^{Kip2} null mutant mice display severe defects including cleft palate, gastrointestinal abnormalities, renal medullary dysplasia, adrenal cortical hyperplasia and lens cell hyperproliferation. See Yan, et al., Genes Dev. 11:973-983 (1997), Zhang, et al., Nature 387: 151-158 (1997). In addition, p57^{Kip2}, together with p21^{Cip1} is also involved in differentiation of myotubes and cells of the lung. See Zhang, et al., Genes Dev. 13: 213-224 (1999). In contrast, with the exception of abnormal maturation of retina amacrine interneurons, CNS-related deficiencies have not been reported in p57 ^{Kip2} gene targeted mice. Dyer, et al., Development 127: 3593-3605 (2000).

The disclosure that follows elaborates upon several of the issues raised supra, including a determination of genes that are regulated by Nurr1. Further aspects of the invention involve a determination of factors involved in the mechanisms of DA cell maturation.

These, and other features of the invention, will be elaborated in the disclosure which follows.

### DETAILED DESCRIPTION OF PREFERRED EMBODIMENTS

### EXAMPLE 1

Choi, et al., Proc. Natl. Acad. Sci. USA 89: 8943-8947 (1992), incorporated by reference, describe a dopamine synthesizing neuronal cell line, referred to as MN9D. Overexpression of Nurr1 in MN9D cells results in cell cycle arrest and morphological maturation characterized by a flattened cell morphology, extension of long neurites and increased dopamine synthesis. A clone of this line was developed, in which expression of Nurr1 was under the control of tetracycline. This line is referred to as “MN9D-Nurr1^{Tet-On}”. The clone was developed by cotransfecting MN9D cells with plasmids pTRE₂-Nurr1, and pTK-Hygro. The first plasmid contained cDNA encoding Nurr1, in host vector PTRE₂. Cotransfection was carried out in accordance with Castro, et al., J. Biol. Chem 276: 43277-43284 (2001), incorporated by reference, in MN9D cells expressing the reverse tetracycline controlled transactivator. Cells were cultured in the presence of doxycycline (“dox”), a tetracycline derivative (2 μg/ml).

Within 24 hours of the treatment with doxocycline, the MN9D-Nurr1^{Tet-On} cells had accumulated at the G1 phase of the cell cycle.

A mature morphological phenotype was evident in the cells after 48 hours, including nuclear localization for Nurr1 in the dox treated cells, as determined by immunocytofluorescence. In brief, an anti-Nurr1 antibody, as described by Wallén, et al., supra, was used, as primary antibody, together with an anti-IgG antibody conjugated to a commercially available fluorophore. Cells were fixed with PFA, and sections were blocked in PBS/0.5% FBS/0.3%-Triton, and then incubated successively with primary antibody (4° C., for 16 hours), and secondary antibody (for 1 hour, at room temperature).

### EXAMPLE 2

These experiments describe a search for genes which influenced the differentiation of the MN9D cells.

To do this, total RNA was isolated from dox treated, MN9D-Nurr1^{Tet-On} cells using standard methods, and reverse-transcribed to cDNA, again following standard methods. RT-PCR was then carried out, using the following primers:

| | | |
|---|---|---|
| tttaccctcg | aagccgaag | (SEQ ID NO: 2) |
| tgtatgctaa | gcgcagaac | (SEQ ID NO: 3) |
| (for Nurr1); | | |
| cggtggaact | ttgacttcgt | (SEQ ID NO: 4) |
| gagtgcaaga | cagcgacaag | (SEQ ID NO: 5) |
| (for p21^{Cip1}) | | |
| ccgaggagga | agatgtcaaa | (SEQ ID NO: 6) |
| aaattccact | tgcgctgact | (SEQ ID NO: 7) |
| (for p27^{Kip1}); and | | |
| gagagaactt | gctgggcatc | (SEQ ID NO: 8) |
| gctttacacc | ttgggaccag | (SEQ ID NO: 9) |
| (for p57^{Kip2}). | | |

In addition, commercially available primers were used for G3PDH. The RT-PCR was carried out for each reaction at 94° C. for 3 minutes, then a varying number of cycles of 94° C., for 30 seconds, 54° C. for 45 seconds, 72° C. for 1 minute, and then 72° C. for 3 minutes. Varying cycles are used because the mRNA for the various proteins is expressed at different levels in the cells. The G3PDH was amplified for a total of 28 cycles, Nurr1 and p21^{Cip1} for 30 cycles, and p27^{Kip1} and p57^{Kip2} for 33 cycles. The assays were run at 0, 1 and 12 hours following the dox treatment. The cDNA encoding Cip/kip members were positive controls, while the G3PDH assay was carried out because this is a housekeeping gene which serves as an internal control.

The results indicated that mRNA for p27^{Kip1} was not induced at any of the time points measured; however, p21^{Cip1} was upregulated after 1 hour of treatment, and p57^{Kip2} was also markedly upregulated but accumulated first, after 12 hours of treatment. Corresponding increases in the levels of p57^{Kip2} and p21^{Cip1} proteins were also observed.

In further experiments, a subclone of the MN9D line which expressed the tetracycline dependent transcription factor was also treated with dox, in the manner described supra, to determine if the effects on cell cycle arrest and Cki expression were specific, and dependent on Nurr1 expression. This was, in fact conformed. These experiments show that Nurr1 does indeed regulate both p21^{Cip1} and p57^{Kip2}, but by different mechanisms. The relatively late induction of the latter suggests that the interaction with Nurr1 is not direct, but that one or more intermediary steps are involved.

### EXAMPLE 3

These experiments were designed to determine expression of molecules in embryonic ventral midbrains of normal, wild type (Sv126/C57B16) mice. Sagittal sections were taken from embryonic day 13.5 mice, and were assayed via in situ hybridization assays, using standard methods, and digoxygenin labeled riboprobes, in accordance with Wallén, et al, supra. There is one exception to this, as p27^{Kip1} analysis was done by immunofluorescence assays. Expression patterns of Nurr1, TH, p21^{Cip1}, p27^{Kip1} and p57^{Kip2} were analyzed.

Strong Nurr1 expression was seen in the ventral midbrain, confirming the results of Zetterström, et al., Mol. Brain Res. 41: 111-120(1996). At this point in time (E13.5), p21^{Cip1} was rather universally distributed in the entire CNS, but p57^{Kip2} showed a distinct expression pattern, and was localized predominantly in the ventricular mitotically active cells which line the ventricles. It was also detected in the ventral midbrain, in a pattern that was virtually indistinguishable from the pattern shown by TH. It overlapped with the Nurr1 expression domain, thus indicating that it is expressed in differentiating DA cells. In contrast to this, p27^{Kip1} expression was not detected at this time point. Both immunohistochemical and confocal imaging of coronal sections from E13.5 ventral midbrains, confirmed that p57^{Kip2} and Nurr1 were coexpressed in developing DA cells, and it was noted that there was a relatively transient expression of p57^{Kip2} mRNA found during a critical stage of DA cell differentiation, i.e., from E13 until E16.5.

### EXAMPLE 4

These experiments were designed to determine if Nurr1 regulates p57^{Kip2} expression in developing DA neurons. In situ hybridization assays, as described supra were carried out, on wild type and Nurr1 null mutant mice. See Zetterstrom et al., supra. Coronal sections were taken, at E13.5. The p57^{Kip2} mRNA levels were drastically diminished in the mantle zone of the null mice, which is where both Nurr1 and TH are expressed in differentiating DA cells, in wild type embryos. The p57^{Kip2} was selectively down-regulated in the mantle zone, but remained at normal levels, in the adjacent ventricular zone. Reduced levels of p57^{Kip2} expression were not due to cellular deficiencies, since other DA markers, such as En1/En2, Raldh 1 and Ptx3 remained at normal expression levels at this stage of development.

These results thus define Nurr1 as being essential for p57^{Kip2} expression, in maturing, postmitotic DA cells.

### EXAMPLE 5

Castro, et al, supra, have shown that Nurr1 induces morphological differentiation of MN9D cells, which are characterized by long and commonly bipolar neurites. The experiments described herein were designed to determine if p57^{Kip2} was functionally important in DA cell maturation.

To test this, MN9D cells were cotransfected, with an expression vector encoding enhanced green fluorescent protein (EGFP), and one or both of Nurr1 and p57^{Kip2}, using standard protocols, such as those described supra. The number of cells which expressed EGFP were counted, three days after the transfection experiments, and were deemed to be differentiated, in accordance with Castro, et al, supra.

The results indicated that while overexpression of p57^{Kip2} was insufficient to promote cell differentiation, expression of p57^{Kip2} and Nurr1 resulted in drastic potentiation of cell differentiation.

### EXAMPLE 6

These experiments were designed to determine if there was some direct interaction between p57^{Kip2} and Nurr1. To test this, human embryonic kidney 293 cells were used. These were transfected with either “Flag” immunotagged Nurr1 using pCMX-Flag-Nurr1, HA-immunotagged p57^{Kip2} using pCMV-HA-p57^{Kip2}, described by Reynaud, et al, Mol. Cell Biol 19:7621-7629 (1999), incorporated by reference, or both. Transfection protocols followed Castro, et al, supra. As a control, empty vector was used.

Following the transfections and cultivation of the cells, nuclear protein extracts were obtained in accordance with Dignam, et al Nucleic Acids Res 11:1475-1489 (1983), and were then resolved on SDS-PAGE. Nuclear cell extracts were immunoprecipitated using anti-Nurr1 or anti-p57^{Kip2} antibodies. These were then immunoblotted with commercially available anti-FLAG and anti-HA antibodies, in accordance with Joseph, et al, Oncogene 20:2877-2888 (2001). The results indicated that the proteins interact physically.

In a follow up to these experiments, a gel shift experiment was carried out, in accordance with Castro, et al, supra, using:

| | |
|---|---|
| agcttgagtt ttaaaaggtc atgctcaatt t, | (SEQ ID |
| | NO: 10) |

and its ³²P labeled complement NBRE (a specific Nurr1 DNA binding site) probe. This is a defined, Nurr1 binding site. The sequence “aaaggtca” was particularly important. Bands were visualized by autoradiography. A shift in Nurr1, and a supershift in Nurr1/p57^{Kip2} complexes bound to NBRE was seen. In further experiments, the combination of the extract with HA specific antibodies abolished the binding of p57^{Kip2} to Nurr1 bound to NBRE probe.

In another experiment to determine if there was direct interaction between p57^{Kip2} and Nurr1, cells from the ventral midbrain of day 15 rat embryos were used. Total cell extracts were prepared according to procedures discussed supra. These were then immunoprecipitated using anti-Nurr1 or anti-IgG (control) antibodies. The resulting immunocomplexes were immunoblotted using anti-p57^{Kip2} antibodies in accordance with protocols discussed supra. Nuclear cell extract from HEK-293 cells transfected with expression vectors encoding p57^{Kip2}, as described supra was used as a control. These results also indicated physical interaction between the proteins.

These experiments suggested that p57^{Kip2} can modulate Nurr1 transcriptional activity. This is discussed in the example which follows.

### EXAMPLE 7

In order to confirm the interaction between p57^{Kip2} and Nurr1, a mammalian two-hybrid assay was performed. HEK-293 cells were co-transfected with expression vectors VP16-p57^{Kip2} and Gal4 DBD-Nurr1 (1-262) either alone or together, and with a luciferase reporter gene driven by four UAS Gal4 binding sites which were cloned upstream of the H. simplex thymidine kinase gene minimal promoter (See Perlmann et al., Genes Dev 9, 769-82 (1995)), using methods set forth in Castro, et al., supra. VP16-p57^{Kip2} encodes the VP16 transcriptional transactivation domain from herpes simplex virus, from pCMX-VP16, followed by the full length, in frame, cDNA sequence of the mouse p57^{Kip2} Gal4 DBD-Nurr1 (1-262) encodes the first 262 amino acid residues of Nurr1 in frame with the yeast Gal4 DNA-binding domain (residues 1-147) of the pCMX-Gal4 vector. The Gal4 DBD-Nurr1 plasmid was used because it activates the reporter gene due to the presence of a transactivation domain within the Nurr1 amino terminal domain. Additionally, plasmids were used which encoded VP16 alone, and Gal4 DBD alone.

The cells were then harvested and analyzed according to Perlmann et al., supra. After normalization to β-galactosidase activities, relative light units (RLU) were computed. The results showed that Gal4 DBD-Nurr1 (1-262) activated the reporter gene, as expected. Activation of the reporter gene was strongly enhanced by co-transfection with VP16-p57^{Kip2}. These results also indicated physical interaction between p57^{Kip2} and Nurr1 occurs.

### EXAMPLE 8

This next experiment was designed to determine if either p21^{Cip1} or p27^{Kip1} interact with Nurr1. HEK-293 cells were transfected with expression vectors encoding either HA-p21^{Cip1} or HA-p27^{Kip1}. An empty expression vector was used as a control. Transfection was performed according to standard protocol discussed supra. To ensure the transfection procedure was successful, following transfection and culturing of the cells, nuclear cell extracts were obtained in accordance with Dignam et al, supra, and resolved on SDS-PAGE. They were then immunoblotted with commercially available anti-p21 and anti-p27 antibodies in accordance with Joseph et al., supra.

HEK-293 cells were then co-transfected with either the expression vector encoding HA-p21^{Cip1} or HA-p27^{Kip1} together with either the empty vector (control) or an expression vector pCMX-Flag-Nurr1, which encodes Flag-Nurr1, i.e., a FLAG tagged version of Nurr1. Nuclear cell extracts were obtained and immunoprecipitated using anti-Nurr1 antibodies. The immune complexes were subjected to immunoblotting using anti-p21^{Cip1} or anti-p27^{Kip1} antibodies. All protocols followed those discussed supra. The results indicated that neither p21^{Cip1} nor p27^{Kip1} interact with Nurr1, because no signal was generated when the immunoblots were probed with anti-p21^{Cip1} or anti-p27^{Kip1}.

### EXAMPLE 9

These experiments were designed in order to determine what type of influence p57^{Kip2} had on Nurr1 transcriptional activity.

MN9D cells were first transfected with a luciferase reporter plasmid which contained three copies of NBRE, referred to as “NBRE-tk-luc,” as described by Perlmann, et al, supra, and either a vector expressing Nurr1, or with one expressing p57^{Kip2}. The cells were harvested 24 hours after transfection and culture, cell extracts were taken, and these were then assayed for luciferase activity, and β-galactosidase activity, as a control.

The results indicated that p57^{Kip2} exerted a negative influence on reporter gene activity in a dose dependent manner. In a control experiment, p57^{Kip2} did not inhibit a retinoic acid receptor dependent reporter gene.

These results lead to the conclusion that Nurr1 and p57^{Kip2} cooperate in inducing maturation of the MN9D cells via a mechanism which may depend on direct interaction between the two proteins.

### EXAMPLE 10

This example describes experiments designed to elucidate the structural features of the protein-protein interaction discussed supra.

Three truncation derivatives of Nurr1 were prepared, using the vectors pCMX-Nurr1¹⁻³⁵⁵, pCMX-Nurr1⁹⁴⁻⁵⁹⁸ and pCMX-Nurr1¹⁸³⁻⁵⁹⁸. The first deletes the carboxy terminal LBD/AF2 domain, and the remaining two lack the first amino terminal 93 and 182 amino acid residues of the AF1 transactivation domain, respectively. These vectors, as well as vectors encoding Nurr1, and HA-p57^{Kip2} were used to transfect HEK293 cells, as described supra. All cells were cotransfected with the HA-p57^{Kip2} vector, and one of the Nurr constructs or control vector.

Following cultivation, co-immunoprecipitation assays were carried out whereby protein sepharose-precleared nuclear extracts were incubated, with antibodies against Nurr1 (See Wallén, et al., supra) and against HA. The extracts were incubated with antibody in nuclear extract buffer overnight, at 4° C. Immunocomplexes which bound to protein A or protein G sepharose were collected via centrifugation and washed, three times, in RIPA buffer.

The results indicated that deletion of the carboxy terminal LBD/AF2 domain did not influence the interaction with p57^{Kip2}, but both the short and long deletions in the amino terminal domain abolished interaction completely.

In follow up experiments, MN9D cells were cotransfected with the EGFP vector described supra, and one of the four Nurr1 vectors described, either alone or together with the p57^{Kip2} expression vector to investigate the effects of Nurr1 and the three Nurr1 truncation derivatives in the maturation of the cells. Differentiation was assayed after 3 days, as described supra.

The Nurr1⁹⁻⁵⁹⁸ and Nurr1¹⁸³⁻⁵⁹⁸ derivatives did not cooperate with p57^{Kip2} in inducing maturation; however, the deletion of the carboxy terminus had no influence on the cooperativity and maturation. The three derivatives in addition to Nurr1, when expressed alone, did induce MN9D cell maturation, but at reduced levels. Thus, achieving the maximal level of differentiation required expression of both p57^{Kip2} and a Nurr1 derivative capable of interacting with p57^{Kip2}.

It was also observed that the Nurr1⁸³⁻⁵⁹⁸ derivative induced expression of p57^{Kip2} as well as did wild type Nurr1. This indicates that the inability of this derivative to functionally cooperate with the p57^{Kip2} in the cell maturation, is not due to inactivation of other essential Nurr1 functions.

### EXAMPLE 11

In view of the observed effect on maturation induced by p57^{Kip2} and Nurr1 coexpression, experiments were carried out to determine what contribution to cell maturation was provided by p57^{Kip2}

To test this, MN9D cells were cotransfected with the expression vectors for EGFP and Nurr1, described supra, either alone or together with an antisense construct, pCMX-asp57^{Kip2}. This antisense construct was obtained by inserting the NcoI-HindIII fragment from pEX10X-p57^{Kip2} into expression vector pCMX in antisense orientation, at its EcORI site. The same system for determining maturation as is described, supra, was used. Expression of “asp57” RNA, i.e., the antisense construct, abolished cell maturation induced by Nurr1. It also inhibited protein expression of endogenous p57^{Kip2}, as well as p57^{Kip2} expressed from a cotransfected expression vector.

In a further experiment, transfected cells were harvested after 24 hours and analyzed by FACS as described by Joseph et al., Oncogene 21:65-77 (2002), incorporated by reference. Cells were sorted by EGFP expression and their distribution in different phases of the cell cycle was determined by quantification of DNA. Asp57 expression didn't disrupt cell cycle arrest induced by Nurr1, presumably due to Nurr1-induced expression of p21^{Cip1} as described supra. Thus these data demonstrate that maturation and cell cycle arrest are independently controlled in these cells. The ramifications of this observation are elaborated in the examples which follow.

### EXAMPLE 12

This experiment was carried out to further investigate specific requirements for cell maturation. A mutated derivative of p57^{Kip2} (p57CK^{mut}) as described in Watanabe, et al. Proc Natl Acad Sci USA 95, 1392-7 (1998) was used, because p57CK^{mut} is unable to inhibit CDK activity. MN9D cells were transfected with expression vectors encoding EGFP and either Nurr1 or Nurr1¹⁻³⁵⁵, either alone or together with expression vectors for p57^{Kip2} or p57CK^{mut}. Transfection was performed via methods set forth supra. Expression of p57CK^{mut} is unable to induce cell cycle arrest but p57CK^{mut} retained the ability to cooperate with Nurr1 in the maturation of MN9D cells. These data indicate that p57^{Kip2} promotes DA cell maturation by a mechanism that is independent of its ability to inhibit CDK activity. It also further supports the view that p57^{Kip2} cooperates in MN9D cell maturation via a mechanism involving direct interaction with Nurr1.

### EXAMPLE 13

The results secured in the preceding experiments suggested that p57^{Kip2} might be involved in DA cell development. To examine this, p57^{Kip2} null mice were used (See Yan et al., supra). Specifically, coronal sections were taken from embryonic day 13.5 and 18.5 mice, as described, supra, and analyzed, also as described.

At E13.5, midbrain DA cells appeared normal in the null mice embryos, based upon the expression of DA neuron marker genes, cell proliferation, and apoptosis.

By E18.5, however, the absence of p57^{Kip2} resulted in drastic reductions in TH immunoreactivity in the ventral midbrain. The TH expression was not affected in other regions of the brain, where catecholaminergic neurons are located, including the locus coeruleus and olfactory bulb. Hence, the phenotype is selective to midbrain DA neurons.

In situ hybridization assays for Nurr1 were carried out, and it was ascertained that Nurr1 expression was diminished in the ventral midbrain of the null mice E18.5 embryos, but was normal in other regions.

In addition, expression of TH and Nurr1 was especially weak in lateral regions of the ventral midbrain, which suggests that TH and Nurr1 expressing cells remain in a medial location in mutant brains.

These data provide support that the onset of p57^{Kip2} expression occurs during late midgestation.

### EXAMPLE 14

The previous experiments showed the importance of p57^{Kip2} in MN9D cells. The expression of this protein during critical stages of DA cell differentiation, in vivo, suggested additional experiments to investigate whether p57^{Kip2} plays a role in DA cell development. More specifically, this experiment was carried out to investigate p57^{Kip2} expression in CSM14.1 cells.

CSM 14.1 is a cell line established from the rat embryonic ventral midbrain and immortalized with the temperature sensitive large T-antigen (See Durand et al., Neurosci. Abstr. (1990)). These cells were used because they resemble immature neural progenitor cells and can be induced to differentiate to a mature DA cell phenotype when grown at 390 C in low concentrations of serum.

CSM 14.1 cells were maintained in DMEM-Glutamax I supplemented with 10% FBS, 100 U/ml penicillin and 100 g/ml streptomycin at 33° C. (a permissive temperature) in 5% CO₂. To induce cell differentiation, FBS was reduced to 1% and the temperature was raised to 39° C. (a non permissive temperature), Durand et al., supra; Haas et al., J. Anat. 20:61-69 (2002). In both cases, cells were cultured for 4 days.

Total cell extracts were obtained from the cells according to standard methods and resolved on SDS-PAGE. These were then immunoblotted with commercially available, anti-Nurr1 or anti-p57 antibodies or antibodies directed against the general neuronal marker NeuN. Filters were subjected to Ponceau staining to ensure equal loading. The results demonstrated that Nurr1, p57^{Kip2} and NeuN are induced as cells differentiate.

In a separate experiment, samples of cells were transfected with expression vector pCMX-asp57^{Kip2}, referred to supra, and cultured for 4 days at either 33° C. (10% FBS) or 39° C. (1% FBS). As a control, an empty expression vector was used. Again, the cells were cultured for 4 days. Transfection with pCMX-asp57^{Kip2} inhibited the acquisition of a mature phenotype, as indicated by lower levels of NeuN. This shows that pCMX-asp57^{Kip2} is important in cell differentiation.

### EXAMPLE 15

These experiments were designed to analyze occurrence of cell death in the ventral midbrains of p57^{Kip2} null mice (E18.5 embryos).

In situ, nuclear DNA fragmentation assay was carried out on cells which had been pretreated for TH immunodetection, in accordance with Joseph, et al, Oncogene 21:65-77 (2002), incorporated by reference. Positive cells were counted individually, by two different individuals.

An increase in apoptotic cells in the entire dopaminergic area was observed in the null mice, as compared to wild type. Indeed, the increase was more than twofold. The increase was specific to the midbrain dopaminergic area, and was not observed elsewhere, leading to the conclusion that there is a strict requirement for p57^{Kip2} in the normal development of midbrain DA cells.

The preceding disclosure describes how Nurr1 is essential for expression of p57^{Kip2}, which is essential to the process of DA neuron differentiation and maturation. Hence, one feature of the invention relates to a method for inducing DA neuron differentiation or maturation, via administration of Nurr1 or a derivative thereof. By “derivative thereof” is meant molecules which lack at least 70 and no more than 300 amino acids of the amino acid sequence of Nurr1 set forth in SEQ ID NO: 1. More preferably, “derivative thereof” means molecules which lack at least 80 and no more than 275 amino acids of the amino acid sequence of Nurr1 set forth in SEQ ID NO: 1, and most preferably molecules which lack at least 90 and no more than 250 amino acids of the amino acid sequence of Nurr1 set forth in SEQ ID NO: 1. The deletion preferably occurs at the carboxy end of the Nurr1 molecule.

The derivatives of the invention are at least 70% homologous to the amino acid sequence set forth in SEQ ID NO: 1. Preferably, a derivative is at least 85% homologous, and more preferably at least 90% homologous to the amino acid sequence set forth in SEQ ID NO: 1. Most preferably, a derivative is at least 95% homologous to the amino acid sequence set forth in SEQ ID NO: 1. “Homology,” as used herein, is defined as being identical to a certain extent to SEQ ID NO: 1, with the remainder of the molecule subject to conservative substitution. By “conservative substitution” is meant that an amino acid present in SEQ ID NO: 1 may be replaced by an amino acid that does not change the function of the molecule, i.e., its ability to interact with p57^{kip2} Such substitutions are known to the skilled artisan. For example, if Glycine is present at a particular point in the molecule, substitution by Alanine is deemed conservative substitution, since the substitution would not be expected to impact function. In contrast, if the position is occupied by Cysteine, due to free sulfhydryl groups, substitution by Alanine would, in fact, be expected to impact function. Other examples, such as the substitution of Leucine by Isoleucine, and vice versa, and others, will be known to the skilled artisan, and will not be repeated here.

Exemplary of such derivatives are truncation variants which lack the carboxy end of the molecule, such as the Nurr1 derivative which consists of only amino acids 1-355 of Nurr1, as described supra. Other variants can be identified and used by the skilled artisan, using the methodologies described in the examples. The Nurr1 or derivatives thereof may be administered, e.g., in the form of a polypeptide per se, or in the form of a recombinant delivery system, as exemplified by the plasmids described in the examples, supra. In addition to neurons, progenitor cells, such as stem cells, can be so treated.

The use of such molecules is envisioned as being useful in the treatment of CNS related disorders, especially those which involve dopamine releasing neurons. Degeneration of such neurons is characteristic of conditions such as Parkinson's disease, while schizophrenia is characterized by an overactive dopaminergic system. Other conditions will be known to the skilled artisan, and need not be set forth here.

The experiments presented supra show that p57^{Kip2} and Nurr1 cooperate in the maturation of the neurons, in a process involving direct physical interaction. Hence, it can be seen that p57^{Kip2} interacts both with CDKs, and cell-type specific transcription factors. Hence, another feature of the invention is a method for regulating p57^{Kip2} by contacting it with a modulating material, such as an agonist or antagonist of the molecule, such as Nurr1 or a derivative thereof.

It is to be noted that p57^{Kip2} promotes differentiation of neurons, after they've exited the cell cycle. The data supra show this.

The data indicate that there is a reciprocal relationship between Nurr1 and p57^{Kip2}. Essentially, Nurr1 activates expression of p57^{Kip2}, and p57^{Kip2} in turn cooperates with Nurr1, as discussed supra. As the examples elaborate, there is probably at least one intermediate step involved in the interaction. What is also clear is that p57^{Kip2} expression in the developing DA cells is dependent upon Nurr1, at a developmental stage when most other analyzed markers, such as En1, En2, Raldh1 and Ptx3 are normally expressed.

The data, supra, do suggest that p57^{Kip2} may have a negative influence on Nurr1's transcriptional activity, possibly in connection with coactivator recruitment. In different promoter contexts, however, it is possible that interaction between the two molecules may exert a positive influence. Notwithstanding this, a further aspect of the invention relates to modulation of Nurr1 activity in a cell by either administering p57^{Kip2} or a portion thereof to Nurr1, so as to inhibit the Nurr1, or conversely to stimulate Nurr1 activity by adding a p57^{Kip2} antagonist, such as an antibody to p57^{Kip2}, a non-functional derivative of Nurr1, and so forth.

It is presumed that the developmental mechanisms described herein are analogous to those in other cell types. For example, some of the requirements for Ckis are independent of their function as CDK inhibitors, as explained in the “Background” section. Results from other systems support this. For example, p57^{Kip2} is known to promote muscle differentiation in a process probably involving transcription factor MyoD. Further, there are redundant activates of p21^{Cip2} and p57^{Kip2} which are required for normal lung alveoli development via mechanisms uncoupled to the control of cellular proliferation. The molecule has also been shown to function in the control of proliferation retinal precursor cells, and in fate determination of a subset of amacrine cells. It has also been observed that a Xenopus homologue of p27^{Kip1} known as p27^{Xic1}, is involved in Muller glial cell differentiation via mechanisms not requiring inhibition of CDKs. It has also been observed that proper development of placental spongiotrophoblasts is disrupted in p57^{Kip2} gene targeted mice, without any measurable increase in CDK activity.

Overall, the characterization of DA neuron development described herein is seen as being useful in the use of stem cells that are specifically designed for therapeutic transplantation in, e.g., Parkinson's disease. For example, See Kim, et al., Nature 418:50-56 (Jul. 4, 2002), incorporated by reference in this regard.

Other features of the invention will be clear to the skilled artisan, and need not be reiterated further.

1. An isolated Nurr1 derivative which lacks at least 70 and no more than 300 amino acids of the amino acid sequence of Nurr1 set forth in SEQ ID NO: 1, wherein said derivative is at least 70% homologous to the amino acid sequence set forth in SEQ ID NO: 1.

2. The isolated Nurr1 derivative of claim 1, which lacks at least 80 and no more than 275 amino acids of the amino acid sequence of Nurr1 set forth in SEQ ID NO: 1.

3. The isolated Nurr1 derivative of claim 1, which lacks at least 90 and no more than 250 amino acids of the amino acid sequence of Nurr1 set forth in SEQ ID NO: 1.

4. The isolated Nurr1 derivative of claim 1, which lacks said amino acids at the C terminus of SEQ ID NO: 1.

5. The isolated Nurr1 derivative of claim 1, wherein said derivative is at least 85% homologous to the amino acid sequence set forth in SEQ ID NO: 1.

6. The isolated Nurr1 derivative of claim 1, wherein said derivative is at least 90% homologous to the amino acid sequence set forth in SEQ ID NO: 1.

7. The isolated Nurr1 derivative of claim 1, wherein said derivative is at least 95% homologous to the amino acid sequence set forth in SEQ ID NO: 1.

8. The isolated Nurr1 derivative of claim 1, which lacks amino acids 1-182 of Nurr1.

9. The isolated Nurr1 derivative of claim 1, consisting of amino acids 1-355 of Nurr1.

10. The isolated Nurr1 derivative of claim 1, which lacks amino acids 1-93 of Nurr1.

11. An isolated nucleic acid molecule which encodes the isolated Nurr1 derivative of claim 1.

12. Expression vector comprising the isolated nucleic acid molecule of claim 1, operably linked to a promoter.

13. Recombinant cell, transformed or transfected with the isolated nucleic acid molecule of claim 1.

14. Recombinant cell, transformed or transfected with the expression vector of claim 12.

15. A method for inducing differentiation or maturation of dopamine producing neurons comprising contacting a dopamine producing neuron or stem cell with Nurr1 or the Nurr1 derivative of claim 1 sufficient to stimulate differentiation or maturation of said neuron or stem cell.

16. The method of claim 15, wherein said Nurr1 derivative lacks a portion of the carboxy terminus of Nurr1.

17. The method of claim 16, wherein said Nurr1 derivative consists of amino acids 1-355 of Nurr1.

18. The method of claim 15, further comprising contacting said dopamine producing neuron or stem cell with p57^{Kip2} sufficient to stimulate differentiation or maturation of said neuron or stem cell.

19. The method of claim 15, comprising inducing differentiation or maturation in a patient with Parkinson's disease.

20-30. (Cancelled).
